(19) United States
(12) Patent Application Publication
(10) Pub. No.: US 2020/0383345 A1
(43) Pub. Date: Dec. 10, 2020

(54) PROCESS FOR PRODUCING AN IMPROVED MESOPHILIC FERMENTED MILK PRODUCT

(71) Applicant: CHR. HANSEN A/S, Hoersholm (DK)

(72) Inventors: Mette Dines CANTOR, Hoersholm (DK); Karin BJERRE, Hoersholm (DK); Helle Skov GULDAGER, Hoersholm (DK); Patrick DERKX, Hoersholm (DK)

(73) Assignee: CHR. HANSEN A/S, Hoersholm (DK)

(21) Appl. No.: 16/643,384

(22) PCT Filed: Aug. 30, 2018
(86) PCT No.: PCT/EP2018/073305

§ 371 Date: Feb. 28, 2020

(30) Foreign Application Priority Data
Aug. 30, 2017 (EP) 17188492.7

(51) Int. Cl. A23C 9/127; A23C 13/16; C12N 1/20

(52) U.S. Cl. CPC A23C 9/127; A23Y 2220/49; C12N 1/20; A23C 13/16

(57) ABSTRACT

The present invention relates to a method for producing a mesophilic fermented dairy product by fermenting a milk substrate with a mesophilic lactic acid bacterium starter culture comprising at least one Lactococcus lactis strain in the presence of at least one Bacillus subtilis subsp. natto or Bacillus coagulans strain.

### FIELD OF INVENTION

The present invention relates to a method for producing a mesophilic fermented dairy product by fermenting a milk substrate with a mesophilic lactic acid bacterium starter culture comprising at least one Lactococcus lactis strain.

### BACKGROUND OF INVENTION

The food industry uses numerous different types of bacteria for preparing food items. For the preparation of fermented dairy products, such as yogurts, cheese or buttermilk, lactic acid bacteria (LAB) are most commonly used. LAB and their metabolic products significantly contribute to the taste and texture of fermented products and inhibit food spoilage by producing considerable amounts of lactic acid.

LABs strains that are currently used by the food industry for preparing fermented dairy products originate from different taxonomical groups, e.g. the genera Streptococcus, Lactococcus, Lactobacillus, Leuconostoc, and Bifidobacterium. The ability of the strains used for fermentation to confer texture to dairy products is to some extent linked to the production of polysaccharides. Not every strain which has been found to have particularly suitable fermentation characteristics, e.g. a good acidification profile, also has good texturizing characteristics. Therefore, it is often required to improve the texture of fermented dairy products.

Different approaches have been used in the prior art for increasing the texture of such products. For example, additives such as gelatin, pectins, alginates, carboxymethyl cellulose, gums, starch, and fiber can be added to the product after its production [1]. However, such additives are generally undesirable in view of the increasing consumer demand for “clean label” products.

Yet another approach for increasing texture is focused on the optimization of the LAB strains used in the fermentation. For example, the use of genetically modified strains with increased galactokinase activity was found to have a significant impact on the texture of products produced with such strains [2]. While these modified strains are highly effective, a high number of consumers tend to prefer naturally occurring strains in dairy products.

The co-fermentation of LABs with bacteria that do not belong to the group of LAB has so far not attracted much attention in the dairy industry. One reason for this resides in the fact that LAB produce high amounts of lactic acid during fermentation which results in a considerable reduction of the pH to 4-5 during fermentation. Most bacteria tolerate only moderate pH reductions which makes them unsuitable for being used in the preparation of fermented dairy products.

Bacteria of the genus Bacillus are not commonly used for fermentation. Nevertheless, there is some evidence that Bacillus strains have been employed in the past for preparing dairy products, such as yogurt. Reference [3] describes the use of Bacillus strains for fermenting milk products such as yogurt in the absence of classical LAB starter cultures.

Reference [4] describes the use of a Bacillus subtilis strain for producing a fermented milk product that might be of therapeutic value. It is reported that antibacterial substances produced by the Bacillus strain provide for a product with long shelf-life and putative therapeutic properties.

Reference [5] describes a method for producing fermented milk using Bacillus subtilis. The method comprises two successive steps. In a first step, milk is fermented with Bacillus subtilis for several hours. In this step, the proteins in the milk are degraded into amino acids or oligopeptides by Bacillus proteases. Subsequently, LAB are added to the milk and the fermentation is continued until the desired pH is reached.

Reference [6] discloses a method for preparing yogurt using levansucrase-producing strains of Bacillus licheniformis or Bacillus subtilis.

Reference [7] describes the preparation of fermented milk products with cheese flavor using a combination of Streptococcus thermophilus and Bacillus stereothermophilus to obtain a product with a cheesy flavor.

Reference [8] is an international patent application which discloses co-fermentation of Streptococcus thermophilus with different Bacillus strains, such as Bacillus subtilis subsp. natto for preparing a thermophilic fermented dairy product.

Finally, Reference [9] reports experiments in which potential effects of contamination by Bacillus subtilis and Bacillus licheniformis on the rheological and textural properties of sour cream have been analyzed.

### SUMMARY OF INVENTION

It has now been surprisingly found in the course of the invention that the texture of mesophilic fermented dairy products can be significantly increased when fermenting a milk substrate with a mesophilic lactic acid bacterium starter culture comprising at least one Lactococcus lactis strain in the presence of at least one Bacillus subtilis subsp. natto or Bacillus coagulans strain.

It appears that strains of these Bacillus species improve the texture conferred to the dairy product by the LAB. The mechanism by which the Bacillus strains exert this effect is unknown. Notably, the shear stress and gel stiffness of products manufactured by the method of the invention is very high and in some cases reaches a level which is fourfold higher than the corresponding shear stress achieved by the same LAB starter culture without the Bacillus strain. In addition, the fermentation of the milk substrate with LAB in the presence of Bacillus has been shown herein to significantly reduce the time that is required for reaching a pH of 4.5. To this extent, the method of the invention aids in the reduction of costs involved with the production process.

In accordance with the above surprising findings, strains of the species Bacillus subtilis subsp. natto or Bacillus coagulans may be used as additives to common mesophilic LAB starter cultures for improving the texture of mesophilic fermented dairy products, e.g. by increasing shear stress or gel stiffness (Complex Modulus). The present invention provides novel fermentation methods using LAB strains and strains of Bacillus subtilis subsp. natto or Bacillus coagulans as well as starter cultures comprising the respective combination of strains.

### DETAILED DISCLOSURE

Therefore, in a first aspect, the present invention relates to a method for producing a mesophilic fermented dairy product, comprising:
(a) providing a milk substrate,
(b) fermenting said milk substrate with a mesophilic lactic acid bacterium starter culture comprising at least one Lactococcus lactis strain,
   wherein step (b) is conducted in the presence of at least one Bacillus strain selected from the group consisting of a Bacillus subtilis subsp. natto strain and a Bacillus coagulans strain.

The invention provides a novel method of manufacturing a dairy product which is based on the mesophilic fermentation of a substrate with LAB in the presence of a Bacillus strain. As used herein, “fermentation” means the conversion of carbohydrates or sugars into alcohols or acids through the action of a microorganism. Preferably, fermentation in the sense of the instant invention comprises the conversion of lactose to lactic acid. The fermentation of carbohydrates or sugars by lactic acid bacteria is particularly preferred.

In the context of the present invention, the term “lactic acid bacterium” designates a gram-positive, microaerophilic or anaerobic bacterium which ferments sugars and thereby produces acids, including lactic acid, acetic acid and propionic acid. Normally, the acid which is predominantly produced is lactic acid. Lactic acid bacteria within the order “Lactobacillales” that have been found useful for industrial purposes include Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Lactic acid bacteria also include the group of strictly anaerobic bifidobacteria, i.e. Bifidobacterium spp. They are frequently used as food cultures alone or in combination with other lactic acid bacteria.

The method of the invention aims at the production of a mesophilic fermented dairy product. A “mesophilic fermented dairy product” is a dairy product which has been prepared by fermentation with mesophilic microorganisms, and in particular mesophilic LAB. “Mesophilic” microorganisms have a growth optimum at moderate temperatures of between 15° C. and 40° C. Typical LAB which are considered mesophilic include, but are not limited to, Lactococcus spp. and Leuconostoc spp. A “mesophilic fermentation” herein refers to fermentation at a temperature between 15° C. and 35° C., preferably between 20° C. and 35° C., and even more preferably between 25° C. and 30° C. Typical dairy products which are considered “mesophilic fermented dairy products” include, but are not limited to, buttermilk, sour milk, cultured milk, smetana, sour cream and fresh cheese, such as quark, tvarog and cream cheese. In contrast, “thermophilic” microorganisms have a growth optimum at temperatures above 43° C. Thermophilic LAB that are used in the dairy industry include, amongst others, Streptococcus spp. and Lactobacillus spp. Accordingly, a “thermophilic fermentation” which is performed with thermophilic microorganisms normally uses a temperature above 35° C. The term “thermophilic dairy product” refers to dairy products prepared by fermentation with thermophilic microorganisms, and in particular thermophilic LAB. However, the thermophilic strains Streptococcus spp. are also used for producing some mesophilic fermented dairy products, e.g. in combination with the mesophilic strains Lactococcus spp., in which case a temperature of e.g. 25° C.-35° C. is preferred, more preferably 30° C.-35° C.

In step (a) of the method of the invention, the milk substrate to be subjected to fermentation is provided. The term “milk substrate” refers to any raw and/or processed milk material that can be subjected to fermentation according to the method of the invention. As used herein, “milk” refers to the lacteal secretion obtained by milking a mammal, such as a cow, a sheep, a goat, a buffalo or a camel. Also included by the term “milk” are protein and/or fat solutions made of plant materials, in particular soy milk. In a preferred embodiment of the present invention, the milk used in the method of the present invention is cow milk.

Useful milk substrates include, but are not limited to, solutions/suspensions of milk or milk-like products comprising protein, such as whole milk or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

The fat content of the milk substrate depends on the specific substrate that is used. In a preferred embodiment of the invention, the method is used for preparing sour cream which means that the milk substrate used in the process is cream having a fat content of 6% to 45%, preferably 9% to 35%, more preferably 12% to 30%, more preferably 14% to 25% and most preferably 16% to 22%.

Prior to fermentation, the milk substrate may be subjected to homogenization or pasteurization. “Homogenization” refers to an intensive mixing to obtain a soluble suspension or emulsion. If homogenization is performed prior to fermentation, it may be performed so as to break up the milk fat globules into globules of smaller sizes to prevent the fat component from separating from the milk. This may be accomplished by forcing the milk at high pressure through small orifices. “Pasteurizing” refers to the treatment of the milk substrate to reduce or eliminate the presence of live organisms, such as microorganisms. Preferably, pasteurization is attained by maintaining the milk substrate at a specified temperature for a specified period of time. The specified temperature is usually attained by heating. The temperature and duration may be selected in order to kill or inactivate certain bacteria, such as harmful bacteria. A rapid cooling step may follow.

In step (b) of the method of the invention, the milk substrate selected for the fermentation process is fermented with a mesophilic lactic acid bacterium starter culture. According to the present invention, a “lactic acid bacteria starter culture” or “lactic acid bacteria starter” is a composition which includes one or more lactic acid bacteria strains that shall be used for the fermentation. A starter culture is normally supplied either as a frozen or freeze-dried culture for bulk starter propagation or as so-called “Direct Vat Set” (DVS) cultures, i.e. a culture intended for the direct inoculation into a fermentation vessel or vat for the production of a dairy product, such as a fermented milk product.

According to the invention, the mesophilic lactic acid bacteria starter culture includes at least one Lactococcus lactis strain. In one embodiment, the Lactococcus lactis strain is a Lactococcus lactis subsp. lactis strain. In another embodiment, the Lactococcus lactis strain is a Lactococcus lactis subsp. cremoris strain.

Apart from the at least one Lactococcus lactis strain, the mesophilic lactic acid bacterium starter culture may include additional mesophilic lactic acid bacteria, such as other strains of L. lactis subsp. lactis or L. Lactis subsp. cremoris. In a particular preferred embodiment, the mesophilic lactic acid bacterium starter culture includes one or more L. lactis subsp. lactis biovar. diacetylactis strains which produces flavor compounds. Alternatively or in addition, the mesophilic starter culture may include one or more bacteria of the following genera: Leuconostoc, Pseudoleuconostoc, Pediococcus or Lactobacillus. Particularly preferred examples include Leuconostoc mesenteroides, Pseudoleuconostoc mesenteroides, Pediococcus pentosaceus, Lactobacillus casei and Lactobacillus paracasei. Particularly preferred examples include Leuconostoc mesenteroides subsp. cremoris, Pseudoleuconostoc mesenteroides subsp. cremoris, Pediococcus pentosaceus, Lactobacillus casei subsp. casei and Lactobacillus paracasei subsp. paracasei.

In a particularly preferred embodiment of the invention, the mesophilic lactic acid bacterium starter culture does not comprise a lactic acid bacterium that produces exopolysaccharides (EPS). In particular, the mesophilic lactic acid bacterium starter culture does not comprise a Streptococcus strain, such as a Streptococcus thermophilus strain.

According to the invention, fermentation of the milk substrate with the mesophilic lactic acid bacterium starter culture is performed in the presence of at least one Bacillus strain selected from the group consisting of a Bacillus subtilis subsp. natto and a Bacillus coagulans strain. Bacillus is a genus of Gram-positive, spore-forming bacteria which have attracted attention during the last years also in the food industry. Bacillus subtilis subsp. natto is known as a nonpathogenic bacterium which is utilized for manufacturing the traditional Japanese fermented soy food “natto”. Bacillus subtilis subsp. natto has received GRAS notification (“Generally Recognized as Safe”) by the FDA and can be purchased from different manufacturers. Bacillus coagulans has been used as a probiotic for its purported support of good digestive and immune health. It is used in some foods, including baked goods, dairy products, and grain products. Bacillus coagulans has also received GRAS notification by the FDA. Strains of Bacillus coagulans are commercially available from different manufacturers.

According to a particularly preferred embodiment of the invention, the Bacillus subtilis subsp. natto strain used during the fermentation with the mesophilic lactic acid bacterium starter culture is selected from the group consisting of DSM 32588, DSM 32589, DSM 32606 and a mutant of one of these deposited strains which have been obtained by using one of the deposited strains as a starting material.

In a particular embodiment of the invention, the classification of a bacterium as a Bacillus subtilis subsp. natto strain according to the present invention is carried out genome sequencing.

In a particular embodiment of the invention, the classification of a bacterium as a Bacillus coagulans strain according to the present invention is carried out genome sequencing.

The term “mutant” refers to a strain which is derived from one of the deposited strains disclosed herein by means of, e.g., genetic engineering, radiation and/or chemical treatment. It is preferred that the mutant is a functionally equivalent mutant, i.e. a mutant that has substantially the same or improved properties with respect to texture, shear stress, viscosity, viscoelasticity and/or gel stiffness as the deposited strain from which it was derived. Especially, the term “mutant” refers to strains obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood as one mutagenization step followed by a screening/selection step), but it is presently preferred that no more than 20, or no more than 10, or no more than 5, treatments (or screening/selection steps) are carried out. In a presently preferred mutant less than 1%, particularly less than 0.1%, less than 0.01%, more particularly less than 0.001%, and most particularly less than 0.0001% of the nucleotides in the bacterial genome have been replaced with another nucleotide, or deleted, compared to the mother strain. In a presently preferred mutant less than 50, particularly less than 30, more particularly less than 20, more particularly less than 10, and most particularly less than 5 the nucleotides in the bacterial genome have been replaced with another nucleotide, or deleted, compared to the mother strain.

While the milk substrate is to be fermented with the mesophilic lactic acid bacterium starter culture in the presence of the at least one Bacillus strain, it will not be necessary that the Bacillus strain is present during the complete fermentation time. It is sufficient that the at least one Bacillus strain is present for a substantial part of fermentation, e.g. for at least 50%, at least 60%, at least 70%, at least 80%, at least 90%, or at least 95% of the overall fermentation time. As used herein, “fermentation time” defines the time period between inoculation of the milk substrate and reaching the pre-determined pH.

For example, the milk substrate may be inoculated with the mesophilic lactic acid bacterium starter culture, followed by incubation of the milk substrate for several hours, e.g. for 1-5 hours, such as for 2, 3 or 4 hours. Subsequently, the one or more Bacillus strains can be added to the milk substrate and fermentation can be continued for several hours until the desired pH has been reached. Conversely, the milk substrate may firstly be inoculated with the one or more Bacillus strains and incubated for several hours, preferably 1-5 hours, such as 2, 3 or 4 hours, followed by the addition of the mesophilic lactic acid bacterium starter culture. The successive inoculation of the milk substrate can be used as a means for adjusting the desired texture of gel stiffness.

In a particularly preferred embodiment, the bacteria from the mesophilic lactic acid bacterium starter culture and the one or more Bacillus strains are present in the milk substrate for the complete fermentation time which means that the mesophilic lactic acid bacterium starter culture and the one or more Bacillus strains are inoculated together into the milk substrate at the start of fermentation.

Typically, the milk substrate, e.g. the cream for preparing sour cream, is inoculated with the mesophilic lactic acid bacterium starter culture so as to achieve a concentration of viable lactic acid bacteria in the milk substrate in the range of 10⁴ to 10¹² cfu (colony forming units) per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable lactic acid bacteria in the milk substrate, e.g. the cream for preparing sour cream, can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

Where the mesophilic lactic acid bacterium starter culture comprises a mixture of two or more different bacteria, it is preferred that the milk substrate, e.g. the cream for preparing sour cream, is inoculated to achieve a concentration of the Lactococcus lactis strains in the milk substrate of at least about 10³ cfu per ml of the milk substrate, at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, or at least about 10⁸ cfu per ml of the milk substrate.

The starter culture may comprise as further components cryoprotectants and/or other conventional additives such as, colorants, yeast extract, sugars and vitamins.

The Bacillus subtilis subsp. natto or a Bacillus coagulans strain will be inoculated into the milk substrate, e.g. the cream for preparing sour cream, such that after inoculation the concentration of the Bacillus strain will be comparable to that recited above in the context of the mesophilic lactic acid bacterium starter culture. This means that the milk substrate, e.g. the cream for preparing sour cream, is inoculated with the one or more Bacillus strains so as to achieve a concentration of viable Bacillus bacteria of the recited species in the milk substrate in the range of 10⁴ to 10¹² cfu per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable Bacillus bacteria of the recited species in the milk substrate can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

It is particularly preferred that the one or more Bacillus strains are added to the milk substrate in a concentration of 10⁷ to 10⁸ cfu/ml of the milk substrate. In another preferred embodiment, the Bacillus strain used in the method of the present invention produces significant amounts of vitamin K.

It is yet not completely understood how the Bacillus species influence the texturizing properties of the lactic acid bacteria. It however appears that the Bacillus strains do not propagate during fermentation to a significant extent. However, it has been shown herein that a significant growth of the Bacillus strains is not required for exerting the positive influence on LAB fermentation.

After inoculation with the mesophilic lactic acid bacterium starter culture and the one or more Bacillus strains, the milk substrate is incubated under conditions suitable for the propagation of the mesophilic lactic acid bacteria. This will preferably include a temperature of between 15° C. and 35° C., more preferably between 20° C. and 35° C., and even more preferably between 25° C. and 35° C., such as between 26° C. and 34° C. The specific temperature to be used during fermentation will mainly depend on the mesophilic fermented dairy product that shall be produced. For example, where the method is applied for the preparation of sour cream, the temperature during the fermentation will be 26° C.-34° C., preferably 28° C.-32° C.

Generally, the fermented dairy product which is produced by the method of the present invention can be any type of dairy product which usually is produced by means of mesophilic fermentation. In a preferred embodiment, however, the mesophilic fermented dairy product is selected from the group consisting of sour cream, sour milk, buttermilk, cultured milk, smetana, quark, tvarog, fresh cheese and cream cheese. In a preferred embodiment, the mesophilic fermented dairy product is sour cream.

The fermentation is carried out until the milk substrate reaches the desired pH which is normally between pH 4.0 and 5.0, and preferably between pH 4.5 and 4.8. Thus, the pH will be monitored during the fermentation process, and the fermentation will be stopped when the pre-determined pH is measured in the fermentation vessel. Depending on the concentration of the starter culture and the product to be manufactured, fermentation may take between 5 and 24 hours, preferably between 5 and 20 hours, more preferably between 5 and 16, more preferably between 5 and 14, more preferably between 6 and 12, more preferably between 7 and 11 and most preferably between 8 and 10 hours.

After fermentation, the fermented dairy product can be cooled and further processed. For example, depending on the type of fermented milk product the processing may include, e.g., the incubation of the product obtained from fermentation with enzymes, such as chymosin and pepsin. When the fermented milk product is a cheese, the processing may also include the cutting of the coagulum into cheese curd particles. The processing of the product may also include the packaging of the fermented milk product. A suitable package may be a bottle, a carton, or the like, having a volume of, e.g. 50 ml to 1000 ml.

The method of the invention has the particular advantage that when using a mesophilic lactic acid bacterium starter culture together with a Bacillus strain selected from the group consisting of a Bacillus subtilis subsp. natto strain and a Bacillus coagulans strain in the preparation of a mesophilic fermented dairy product, such as sour cream, the texture properties of the resulting dairy product, in particular viscosity, shear stress, gel stiffness and gel firmness, can be significantly improved.

Preferably, by using a method as defined herein, an increase in the shear stress, gel stiffness and/or gel firmness of the fermented dairy product of at least, 10%, at least 15%, at least 20%, at least 25%, at least 30%, at least 35%, at least 40%, at least 45%, at least 50%, at least 75%, at least 100%, at least 150%, at least 200%, at least 250%, at least 300%, at least 350%, at least 400%, at least 450%, or at least 500% can be obtained relative to fermentation of the same milk substrate under identical conditions in the absence of any Bacillus strain.

In one preferred embodiment, the increase in shear stress of a fermented dairy product is at least 5, at least 10, at least 15, at least 20, at least 25, or at least 30 Pa relative to a corresponding fermented dairy product obtained by fermentation of the same milk substrate under identical conditions in the absence of any Bacillus strain.

In another preferred embodiment, the increase in the gel stiffness of a fermented dairy product is at least 25, at least 50, at least 100, at least 150, at least 200, at least 250 Pa, or at least 300 Pa, relative to a corresponding fermented dairy product obtained by fermentation of the same milk substrate under identical conditions in the absence of any Bacillus strain.

In yet another preferred embodiment, the increase in gel firmness of a fermented dairy product is at least 50 (g×sec), at least 75 (g×sec), at least 100 (g×sec), at least 125 (g×sec), at least 150 (g×sec), at least 175 (g×sec), or at least 200 (g×sec).

In a particular embodiment of the invention, shear stress is measured by the method defined in Example 1.

In a particular embodiment of the invention, gel stiffness is determined as Complex Modulus using the method defined in Example 1.

In a particular embodiment of the invention, gel stiffness is determined as Positive Compression Area using the method defined in Example 1.

In a particularly preferred embodiment of the invention, the above-described method relates to the manufacturing of sour cream. Accordingly, in a particularly preferred embodiment a method for producing sour cream is provided, said method comprising:
(a) providing cream having a fat content of at least 6%,
(b) inoculating said cream with a mesophilic lactic acid bacterium starter culture comprising at least one Lactococcus lactis strain, and optionally additional mesophilic lactic acid bacteria,
(c) inoculating said cream with at least one Bacillus strain selected from the group consisting of a Bacillus subtilis subsp. natto strain and a Bacillus coagulans strain,
(d) fermenting said cream until the pH reaches 4.0 to 5.0, more preferably 4.5 to 4.6,
(e) obtaining the sour cream.

In a first step of the above method, cream is provided as a milk substrate. The cream used for the process of manufacturing sour cream is preferably obtained from cow milk. The fat content of the cream will be at least 6% which is the usual fat content for cream that is used in the production of sour cream. Typically, the fat content is standardized prior to fermentation to comply with food regulations. During standardization, dry ingredients may be added to the cream such as whey or caseins. If stabilizers are to be added, they may also be added at this stage of the preparation process. Suitable stabilizers include, for example, polysaccharides, starch and gelatin.

Subsequently, the cream is preferably subjected to homogenization in order to break down larger fat globules into smaller globules, thereby providing an even suspension in preventing the separation of the whey. Homogenization of the cream can be carried out in a standard homogenizer which is routinely used in the dairy industry. Homogenization conditions may comprise a pressure of 100 to 200 bar, preferably 130 to 150 bar and a temperature of between 50° C. and 80° C., preferably between 65° C. and 75° C. In a particular embodiment, homogenization is carried out in two steps at 150-200 bar and 65° C. to 75° C. in a first step and at 30-60 bar and 65° C. to 75° C. in a second step.

After homogenization, the cream may undergo pasteurization to kill potentially harmful bacteria. Preferably, pasteurization is carried out as a high temperature short time (HTST) pasteurization, which normally means that the cream is heated to 80° C. to 90° C. and incubated at that temperature for about 2 to 10 minutes, in particular 2 to 5 minutes. After pasteurization, the cream is cooled down to the selected fermentation temperature for inoculation of the mesophilic lactic acid bacterium starter culture.

The cream is then inoculated with a mesophilic lactic acid bacterium starter culture as defined above which comprises at least one Lactococcus lactis strain, and optionally additional mesophilic lactic acid bacteria. Normally the cream is inoculated with 0.01-0.02% starter culture. The inoculated cream is then normally incubated for about 12 to 18 hours until a pH of 4.5 to 4.6 is reached. Once the pre-determined pH is reached, the fermented sour cream product is cooled and packaged.

In a second aspect, the invention relates to a composition for producing a mesophilic fermented dairy product, comprising
(a) a mesophilic lactic acid bacterium starter culture comprising at least one Lactococcus lactis strain, and
(b) a Bacillus strain selected from the group consisting of a Bacillus subtilis subsp. natto and a Bacillus coagulans strain.

The composition can be formulated for being suitable for direct inoculation of a milk substrate or another culture medium prior to fermentation.

In a third aspect, the invention relates to a mesophilic fermented dairy product obtainable by the method described in connection with the first aspect of the invention. Preferably, said fermented dairy product comprises
(a) at least one Lactococcus lactis strain, and
(b) at least one Bacillus strain selected from the group consisting of Bacillus subtilis subsp. natto or Bacillus coagulans strain.

In a fourth aspect, the invention relates to a mesophilic fermented dairy product, comprising
(a) at least one Lactococcus lactis strain, and
(b) at least one Bacillus strain selected from the group consisting of Bacillus subtilis subsp. natto or Bacillus coagulans strain.

The Lactococcus lactis strain which is present in the composition according to the second aspect of the invention or in the mesophilic fermented dairy product according to the third or fourth aspect of the invention is preferably selected from the group consisting of Lactococcus lactis subsp. lactis or Lactococcus lactis subsp. cremoris.

Apart from the at least one Lactococcus lactis strain, the composition according to the second aspect of the invention or the mesophilic fermented dairy product according to the third or fourth aspect of the invention may include additional mesophilic lactic acid bacteria, such as other strains of L. lactis subsp. lactis, L. lactis subsp. lactis biovar, diacetylactis, or L. Lactis subsp. cremoris. Alternatively, the composition according to the second aspect of the invention or the mesophilic fermented dairy product according to the third or fourth aspect of the invention may include mesophilic bacteria of the genus Leuconostoc, Pseudoleuconostoc, Pediococcus or Lactobacillus. Particularly preferred examples include Leuconostoc mesenteroides, Pseudoleuconostoc mesenteroides, Pediococcus pentosaceus, Lactobacillus casei and Lactobacillus paracasei. Particularly preferred examples include Leuconostoc mesenteroides subsp. cremoris, Pseudoleuconostoc mesenteroides subsp. cremoris, Pediococcus pentosaceus, Lactobacillus casei subsp. casei and Lactobacillus paracasei subsp. paracasei.

Likewise, the composition according to the second aspect of the invention or the mesophilic fermented dairy product according to the third or fourth aspect of the invention preferably contains a Bacillus subtilis subsp. natto strain selected from the group consisting of strains DSM 32588, DSM 32589, DSM 32606, and mutant of one of these deposited strains which have been obtained by using one of the deposited strains as a starting material.

The mesophilic fermented dairy product according to the third or fourth aspect of the invention preferably is selected from the group consisting of sour cream, sour milk, buttermilk, cultured milk, smetana, quark, tvarog, fresh cheese and cream cheese, and more preferably is sour cream.

In a fifth aspect, the invention relates to the use of a Bacillus strain selected from the group consisting of a Bacillus subtilis subsp. natto and a Bacillus coagulans strain for increasing the texture, viscosity, viscoelasticity, shear stress, gel stiffness and/or gel firmness of a mesophilic fermented dairy product. Preferably, the mesophilic fermented dairy product is selected from the group consisting of sour cream, sour milk, buttermilk, cultured milk, smetana, quark, tvarog, fresh cheese and cream cheese, and more preferably is sour cream. The Bacillus subtilis subsp. natto strain according to the 5^{th} aspect of the invention is preferably selected from the group consisting of strains DSM 32588, DSM 32589, DSM 32606, and mutant of one of these deposited strains which have been obtained by using one of the deposited strains as a starting material.

The use of the terms “a” and “an” and “the” and the like in the context of describing the invention is to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1: Bacillus subtilis Subsp. natto Reduces Acidification Time and Improves Rheology During Sour Cream Preparation

Strains

Bacillus subtilis natto: DSM 32588

LC starter: Lactococcal starter culture comprising a number of Lactococcus lactis subsp. lactis and a number of Lactococcus lactis subsp. cremoris.

Milk Base

TABLE 1
Composition of milk base (18% fat base)

| | Amount (g) | Protein (%) | Carbohydrate (%) | Fat (%) |
|---|---|---|---|---|
| Mini milk | 1600 | 3.5 | 4.7 | 0.4 |
| 38% cream | 1400 | 2.1 | 3.2 | 38.0 |
| 18% fat base | 3000 | 2.85 | 4.00 | 17.95 |

Process

The milk base in 200 ml bottles was inoculated with the LC starter as acidifier (0.01%) 1) with no Bacillus strain (reference) and 2) with 3 levels of added Bacillus strain (10⁵ CFU/ml, 10⁷ CFU/ml and 10⁸ CFU/ml). Fermentation was carried out at 30° C. with a cold start until a pH of 4.5 was reached. Bottles for measurement of rheology is cooled to 4° C. in water bath and stored at 4° C. until measurement. Fermentations were run in duplicate for each sample and rheology determinations of Complex Modulus and Shear Stress were conducted in duplicate.

Measurements

Acidification

Acidification was measured using a Cinac system.

Complex Modulus and Shear Stress

Two days after production, the fermented milk product was brought to 13° C. and manually stirred gently by means of a spoon (5 times) until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:

Holding Time (to Rebuild to Somewhat Original Structure)

5 minutes without any physical stress (oscillation or rotation) applied to the sample. Oscillation step (to measure the elastic and viscous modulus, G′ and G″, respectively, therefore calculating the complex modulus G*)
Constant strain=0.3%, frequency (f)=[0.5 . . . 8] Hz
6 measuring points over 60 s (one every 10 s)

Rotation step (to measure shear stress at 300 l/s)

Two steps were designed:
1) Shear rate=[0.3-300] 1/s and 2) Shear rate=[275-0.3] 1/s.

Each step contained 21 measuring points over 210 s (on every 10 s).

The shear stress at the peak point of the flow curves was chosen for further analysis.

The Complex Modulus G* is a parameter, which expresses Gel Stiffness.

Positive Compression Area

A back extrusion test was conducted to evaluate gel firmness. The samples were tempered to be 13° C. for one hour prior to shear stress measurements. Stirring with spoon was applied to give a homogenous sample, i.e. stirring five times. Measurement was done by TA-XT plus, software Texture Expert Exceed v6.1.9.0. A cylindrical acrylic probe (Ø40 mm) penetrated the yogurt to a depth of 15 mm with a speed of 2 mm/s and a trigger force of 5 g. The positive area was used as firmness measurement.

Cell Counts

The number of Bacillus cells at the end of the fermentation was determined by plating on TSA (Tryptic Soy Agar) agar plates.

Results

Acidification

TABLE 2
Acidification

| | | Average difference |
|---|---|---|
| | Time to pH | compared to |
| Sample | 4.5 (hours) | reference (hours) |
| LC starter A (Reference) | 18.40 | |
| LC starter B (Reference) | 18.26 | |
| LC starter + Bacillus 10⁸/ml A | 14.67 | −3.7 |
| LC starter + Bacillus 10⁸/ml B | 14.59 | |
| LC starter + Bacillus 10⁷/ml A | 16.92 | −1.6 |
| LC starter + Bacillus 10⁷/ml B | 16.92 | |
| LC starter + Bacillus 10⁵/ml A | 17.84 | −0.4 |
| LC starter + Bacillus 10⁵/ml B | 18.09 | |

It was found that the addition of Bacillus strain DSM 32588 in a concentration of 10⁸ CFU/ml to sour cream milk base that had been inoculated with a multi-Lactococcal starter culture (LC starter) decreased the acidification time by up to 3.7 hours.

As mentioned above the fermentations were carried out with a cold start. This means that the acidification time is considerable longer that what it would have been, if the temperature had been 30° C. from the start of the fermentations. However, it is still possible to compare the performance of the samples of the invention with the reference samples.

Rheology

TABLE 3
Rheology

| | Positive | G* - Complex | Shear |
|---|---|---|---|
| | Compression | Modulus (Pa) | Stress (Pa) |
| Sample | Area (g* sec) | at 2.6 Hz | at 90/s |
| LC starter A (Reference) | 543.37 | 562 | 112 |
| | | 612 | 115 |
| LC starter B (Reference) | 396.72 | 491 | 113 |
| | | 554 | 112 |
| LC starter + Bacillus 10⁸/ml A | 880.05 | 2280 | 147 |
| | | 2040 | 129 |
| LC starter + Bacillus 10⁸/ml B | 959.38 | 2310 | 140 |
| | | 2350 | 145 |
| LC starter + Bacillus 10⁷/ml A | 734.47 | 1520 | 145 |
| | | 1630 | 148 |
| LC starter + Bacillus 10⁷/ml B | 638.54 | 1400 | 153 |
| | | 1340 | 149 |
| LC starter + Bacillus 10⁵/ml A | 404.38 | 617 | 116 |
| | | 716 | 117 |
| LC starter + Bacillus 10⁵/ml B | 424.26 | 656 | 117 |
| | | 672 | 118 |

Conclusion for Rheology

It was found that the addition of Bacillus in amounts of 10⁷ and 10⁸ CFU/ml has a significant effect on the firmness of the sour cream. This is shown by significant increase in positive compression area, complex modulus and shear stress. At an inoculation concentration of 10⁵ CFU/ml Bacillus the texture properties are comparable to the reference and no effect was observed.

Cell Counts

TABLE 4
Cell counts

| | CFU/ml |
|---|---|
| LC starter + Bacillus 10⁸/ml | 9.0E04 |
| LC starter + Bacillus 10⁷/ml | 1.5E04 |
| LC starter + Bacillus 10⁵/ml | 3.0E02 |

It was found that about half of the Bacillus cells survive the fermentation process.

### Example 2: Sour Cream Preparation Using Mesophilic Cultures Comprising a Bacillus subtilis Subsp. natto

This experiment was conducted in the same way as Example 1 with the exception that instead of using a mesophilic culture comprising Bacillus strain DSM 32588 at three concentration levels, two mesophilic cultures containing either Bacillus strain DSM 32588 or Bacillus strain DSM 32589, at a dosage of 10⁸ CFU/ml were tested.

Results

Acidification

TABLE 5
Acidification

| | | Average difference |
|---|---|---|
| | Time to pH | compared to |
| Sample | 4.5 (hours) | reference (hours) |
| LC starter A (Reference) | 19.67 | |
| LC starter B (Reference) | 19.67 | |
| LC starter + Bacillus DSM 32588 10⁸/ml A | 16.51 | −3.16 |
| LC starter + Bacillus DSM 32588 10⁸/ml B | 16.51 | |
| LC starter + Bacillus DSM 32589 10⁸/ml A | 16.76 | −2.91 |
| LC starter + Bacillus DSM 32589 10⁸/ml B | 16.76 | |

It was found that the mesophilic cultures containing either Bacillus strain DSM 32588 or DSM 32589 were both able to decrease the acidification time by approx. 3 hours.

Rheology

TABLE 6
Rheology

| | Positive | G* - Complex | Shear |
|---|---|---|---|
| | Compression | Modulus (Pa) | Stress (Pa) |
| Sample | Area (g* sec) | at 2.6 Hz | at 75.2/s |
| LC starter (Reference) | 384.04 | 486.5 | 104.0 |
| LC starter + Bacillus DSM 32588 10⁸/ml | 464.11 | 991.0 | 127.0 |
| LC starter + Bacillus DSM 32589 10⁸/ml | 438.63 | 756.0 | 129.50 |

It was found that the addition of both Bacillus strain DSM 32588 and DSM 32589 in an amount of 10⁸ CFU/ml has a significant effect on the firmness of the sour cream. This is shown by significant increase in positive compression area, complex modulus and shear stress.

### DEPOSITS AND EXPERT SOLUTION

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on 2017 Aug. 16 under the accession number DSM 32588.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on 2017 Aug. 23 under the accession number DSM 32606.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on 2017 Aug. 16 under the accession number DSM 32589.

The deposits have been made under the conditions of the Budapest Treaty on the International Recognition of the Deposit of Microorganisms for the Purposes of Patent Procedure.

The Applicant requests that a sample of the deposited microorganisms should be made available only to an expert approved by the Applicant.

### REFERENCES

[1] Tasneem et al. (2013 Crit Rev Food Sci Nutr., 54(7):869-79.
[2] WO 2011/026863
[3] US2009/0011081 A1
[4] U.S. Pat. No. 5,077,063
[5] CN103300147 A
[6] CN 103190478 A
[7] U.S. Pat. No. 3,674,508
[8] WO 2017/005601 A
[9] Mehta et al. (2016), Journal of Animal Science, vol. 94 No. supplement 5, p. 264-265

All references cited in this patent document are hereby incorporated in their entirety by reference.

1. A method for producing a mesophilic fermented dairy product, comprising:
(a) providing a milk substrate,
(b) fermenting said milk substrate with a mesophilic lactic acid bacterium starter culture comprising at least one Lactococcus lactis strain,
wherein step (b) is conducted in the presence of at least one Bacillus strain selected from the group consisting of a Bacillus subtilis subsp. natto strain and a Bacillus coagulans strain.

2. The method of claim 1, wherein the fat content of the milk substrate is between 16% and 22%.

3. The method of claim 1, wherein the fermenting in step (b) is performed at a temperature ranging from 25° C. to 35° C.

4. The method of claim 1, wherein the milk substrate is cream.

5. The method of claim 1, wherein said mesophilic lactic acid bacterium starter culture does not comprise an EPS-producing lactic acid bacterium.

6. The method of claim 1, wherein said Bacillus subtilis subsp. natto strain is selected from the Bacillus subtilis subsp. natto strains deposited with the Deutsche Sammlung von Mikroorganismen und Zellkulturen GmbH (DSMZ) under accession number DSM 32588, accession number DSM32589, and accession number DSM 32606.

7. The method of claim 1, wherein said Bacillus strain is added to the milk substrate at a concentration of 10⁷ to 10⁸ CFU/ml milk substrate.

8. The method of claim 1, wherein said Bacillus strain produces vitamin K.

9. The method of claim 1, wherein the method produces a mesophilic fermented dairy product selected from the group consisting of sour cream, sour milk, buttermilk, cultured milk, smetana, quark, tvarog, fresh cheese and cream cheese.

10. The method of claim 1, wherein fermenting step (b) results in an increase of shear stress, gel stiffness and/or gel firmness of at least 10% relative to fermenting the milk substrate with the mesophilic lactic acid bacterium starter culture in the absence of the Bacillus strain.

11. A composition for producing a mesophilic fermented dairy product, comprising:
(a) a mesophilic lactic acid bacterium starter culture comprising at least one Lactococcus lactis strain, and
(b) at least one Bacillus strain selected from the group consisting of a Bacillus subtilis subsp. natto strain and a Bacillus coagulans strain.

12. The composition of claim 11, wherein said Lactococcus lactis strain is selected from the group consisting of Lactococcus lactis subsp. lactis strains and Lactococcus lactis subsp. cremoris strains.

13. The composition of claim 11, wherein said Bacillus subtilis subsp. natto strain is selected from the group consisting of strains DSM 32588, DSM32589, and DSM 32606.

14. A mesophilic fermented dairy product obtained by the method according to claim 10.

15. A mesophilic fermented dairy product, comprising
(a) at least one Lactococcus lactis strain;
(b) at least one Bacillus strain selected from the group consisting of a Bacillus subtilis subsp. natto strain and a Bacillus coagulans strain.

16. The mesophilic fermented dairy product of claim 14, wherein said product is selected from the group consisting of sour cream, sour milk, buttermilk, cultured milk, smetana, quark, tvarog, fresh cheese and cream cheese.

17. The mesophilic fermented dairy product of claim 16, wherein said Bacillus subtilis subsp. natto strain is selected from the group consisting of strains DSM 32588, DSM 32589, and DSM 32606.

18. A method for increasing the shear stress, gel stiffness and/or gel firmness of a mesophilic fermented dairy product, comprising fermenting a milk substrate with a mesophilic lactic acid bacterium starter culture comprising at least one Lactococcus lactis strain, in the presence of at least one Bacillus strain selected from a Bacillus subtilis subsp. natto strain and a Bacillus coagulans strain.

19. A method according to claim 18, wherein said Bacillus subtilis subsp. natto strain is selected from the group consisting of strains DSM 32588, DSM 32589, and DSM 32606.

20. A method according to claim 18, wherein said mesophilic fermented dairy product is selected from the group consisting of sour cream, sour milk, buttermilk, cultured milk, smetana, quark, tvarog, fresh cheese and cream cheese.
